(19) United States
(12) Patent Application Publication
(10) Pub. No.: US 2025/0084360 A1
(43) Pub. Date: Mar. 13, 2025

(54) INCUBATION METHOD AND SAMPLE HOLDER

(71) Applicant: LUMICKS CA HOLDING B.V., Amsterdam (NL)

(72) Inventors: Rui Pedro RIJO DA COSTA CARVALHO, Amsterdam (NL); Marinus Theodorus Johannes VAN LOENHOUT, Amsterdam (NL); Zhongjie YU, Amsterdam (NL); Trillian Ashley GREGG, Amsterdam (NL); Serena Alba DAVOLI, Amsterdam (NL); Mattijs DE GROOT, Amsterdam (NL)

(73) Assignee: LUMICKS CA HOLDING B.V., Amsterdam (NL)

(21) Appl. No.: 18/725,360

(22) PCT Filed: Dec. 29, 2022
(86) PCT No.: PCT/EP2022/088026

§ 371 Date: Jun. 28, 2024

(30) Foreign Application Priority Data
Dec. 31, 2021 (NL) 2030387
May 13, 2022 (EP) 22173281.1
Jun. 1, 2022 (EP) 22176847.6

(51) Int. Cl. C12M 1/00; C12M 1/12; C12M 1/26; C12M 1/34; C12M 3/06

(52) U.S. Cl. CPC C12M 41/14; C12M 23/16; C12M 25/16; C12M 33/04; C12M 41/46

(57) ABSTRACT

A method is provided, comprising the steps of: providing a sample holder (300) having a flow channel (310) extending between a channel inlet (320) at an opening side (OS) and a channel outlet (320) at the opening side (OS) and defining a ceiling (312) associated with the opening side (OS); loading, in an upright orientation of the sample holder (300) in which the opening side (OS) is upward, the flow channel (310) with a liquid containing first cellular objects (330); forming a layer of the first cellular objects on the ceiling, comprising—inverting the sample holder into an upside-down orientation, and—incubating the first cellular objects for a defined period in the upside-down orientation to promote binding of least some of the cellular objects to the ceiling; and arranging, after the incubation, the sample holder in the upright orientation for a further use. Associated sample holders are also provided.

### TECHNICAL FIELD

The present disclosure relates to a method for manipulating cells and associated sample apparatus.

### BACKGROUND

In microfluidic studies and in particular in studies related to biological processes such as cellular avidity, control over interaction and/or incubation time may be of importance. Such studies may involve application of forces, such as centrifugal force or (ultrasonic) acoustic force.

Providing sample holders with a target cell layer support that are compatible with microscopy applications and that remain sealed under centrifugal force while providing convenient sample handling (loading and/or unloading) is currently difficult, and tends to require elaborated manipulation and/or particular sample holder design (see Yang et al. “Multiplexed Single-Molecule Force Spectroscopy Using a Centrifuge.” Nature Communications 7, no. 1 (April 2016): 11026, DOI: 10.1038/ncomms11026).

Also, for cellular avidity applications the contact time (incubation time) between target and effector cells is an important parameter that may be comparably short and/or needs to be tightly controlled (e.g. using precisely 5 minutes of incubation for each experimental run, in order to obtain reproducible results).

Further, it is advantageous if plural samples can be studied in parallel. These requirements conflict with complicated manipulation.

Hence, improvements regarding simplification and/or cost reduction are desired.

### SUMMARY

Herewith, methods and sample holders are provided as set out below and in particular as specified in the appended claims.

A method comprises the steps of:
providing a sample holder having a flow channel extending between a channel inlet at an opening side and a channel outlet at the opening side and defining a ceiling associated with the opening side and a floor opposite the ceiling;
loading, in an upright orientation, the flow channel with a liquid containing first objects;
forming a layer of the first objects on the ceiling and/or floor, comprising inverting the sample holder into an upside-down orientation, and causing binding of at least some of the objects to the ceiling and/or floor,
arranging, after the binding, the sample holder in the upright orientation for a further use.

The loading the flow channel may comprise flowing a liquid containing the first objects via the channel inlet into the flow channel, wherein at least a portion of the liquid may be flown through the flow channel and out of the channel outlet. Forming the layer of the first objects on the ceiling may comprise sedimentation of the objects onto the ceiling in the upside-down orientation. Forming the layer on the floor may comprise floating of the objects against the floor. Also or alternatively, forming the layer may comprise subjecting the sample holder and/or the objects to a controllable force urging the objects towards the ceiling or floor, respectively.

The first objects may be beads or other inanimate objects. However, they preferably are, or essentially are or at least comprise, biological objects such as cellular objects, e.g. biological cells, cell groups or few-cell tissue portions. The first objects may also be, or essentially be or comprise subcellular organelles. The liquid preferably is an aqueous liquid, e.g. a biological cell culture medium. Causing the binding may comprise controlling for a defined period conditions of the contents of the flow channel such as temperature and/or lighting, etc.

As an exemplary further use, the sample holder may be subjected to a controllable force, e.g. a centrifugal force and/or at least part of the flow channel may be used for microscopic imaging of flow channel contents, e.g. imaging bound first objects.

In particular, herewith is provided a method, comprising the steps of:
providing a sample holder having a flow channel extending between a channel inlet at an opening side and a channel outlet at the opening side and defining a ceiling associated with the opening side;
loading, in an upright orientation the flow channel with a liquid containing first cellular objects, in particular biological cells such as target cells;
forming a layer of the first cellular objects on the ceiling, comprising inverting the sample holder into an upside-down orientation, and
incubating the cellular objects for a defined period in the upside-down orientation to promote binding of least some of the cellular objects to the surface portion;
arranging, after the incubation, the sample holder in the upright orientation for a further use.

Thus, a layer of the first cellular objects is formed on the ceiling of the channel. The forming may comprise sedimentation of the cellular objects from the sample liquid.

Cellular objects not bound to the ceiling after the predetermined period of the incubation will fall from the ceiling when the sample holder is returned to the upright orientation after the incubation. Unbound cellular objects may be moved away through the channel and/or be removed from the channel, this may prevent that they become bound again to the ceiling and/or affect the layer otherwise. Thus, one or more of the incubation, formation of the layer and composition of the layer may be well controlled. The further use in the upright orientation can then likewise proceed on the basis of selectivity of attachment of the cellular objects to the ceiling.

The method may comprise, during the step of inverting and/or the step of incubating, leaving the channel inlet and/or channel outlet open during the inverting and/or incubating. Then, the method may comprise selecting the liquid and the channel inlet and/or channel outlet to retain the liquid and cellular objects in the flow channel by capillary force during the inverting and/or incubating.

This simplifies manipulation of the sample holder and hence of the method, since closing of the inlet and/or outlet is obviated.

Capillary forces of a liquid in an opening depend, inter alia, on composition and/or surface tension of the liquid, and on one or more of shape, material, structure and/or treatment of a contact surface of the opening being contacted/to be contacted by the liquid. Typical pipetting connectors of glass and/or polymeric microfluidic flow channel chips, such as for biologic and/or “lab on a chip”-type studies, surprisingly prove to be well suited for inverting (“flipping over”) and reorienting (“flipping back”) while retaining aqueous solutions such as biological cell culture media, without requiring closing measures as previously held necessary and applied in customary practice.

The layer may be a first layer. Then, the method may further comprise, after the step of arranging, the further step of loading in the upright orientation the flow channel with a liquid containing second objects, in particular second cellular objects, more in particular further biological cells which may interact with the first cellular objects, such as effector cells binding to target cells. The method may also comprise forming a second layer of the second objects on the first layer and/or the ceiling, comprising inverting the sample holder into the upside-down orientation, Forming the second layer may comprise forming the layer by sedimentation of the second objects onto the first layer and/or the ceiling. The method may also comprise further incubating the second layer for a second defined period in the upside-down orientation to promote binding of least some of the second objects to the first layer and/or the ceiling. The method may also comprise rearranging, after the incubation, the sample holder in the upright orientation.

Thus, a further layer may be formed on the (first) layer, and interaction of the layers may be determined, in particular interaction affecting adhesion to the ceiling or interaction affecting adhesion of the first and second objects.

Similar to the discussion above, chances are reduced that (objects in) the first layer are affected by unbound second objects.

By controlling one or more of incubation conditions, the first and/or second defined period, a time period wherein the sample holder is inverted into the upside-down orientation and/or maintained in the upside-down orientation, interaction between the first and/or second objects and the ceiling, and/or interaction between the first and second objects may be well controlled.

Between the step of arranging the sample holder in the upright orientation and the step of loading the flow channel with a liquid containing second objects, as a further step may be provided: removing unbound cellular objects from the channel, in particular by flushing a liquid through the channel.

This further prevents interaction between bound and unbound objects. Generally, flushing sample liquid so as to remove undesired objects from the flow channel may be referred to as “washing”.

The sample holder may comprise a liquid reservoir fluidly connected with the channel outlet. Then, the method may comprise the further step of: loading with a liquid at least part of the reservoir after the arranging and/or rearranging the sample holder in the upright orientation, in particular loading the reservoir through the flow channel. The reservoir may be open toward the opening side and may have an opening not suitable to retain the liquid and cellular objects in the flow channel and/or reservoir by capillary force during the inverting and/or incubating.

This may facilitate one or more of flushing, washing and incubation. Thus, after formation of the first and/or second layer, and possibly after removal of unbound objects such as first and/or second cellular objects, the further incubation can be applied to a controlled layer (see above). Also, by arranging the sample holder in the upright orientation, modification of the liquid and/or other sample components is facilitated; the modification may comprise one or more of addition, removal, and exchange of liquid and/or other sample components; e.g. nutrients may be provided.

Also or alternatively, this facilitates modification of sample components, in particular addition of sample liquid. For, it has been considered that addition of sample liquid, e.g. dispensing sample fluid into the flow channel from a dispenser such as a pipette, is generally simpler than removal of sample fluid, e.g. aspirating. Thus, administrating sample components is facilitated.

The liquid in the liquid reservoir may further assist as a buffer for processes in (sample liquid in) the flow channel. E.g. having a comparably large reservoir in fluidic contact with the flow channel may provide diffusion of molecules/material between the channel and the fluid reservoir. This can e.g. facilitate keeping the pH in the flow channel with living cells at a desired level and/or it can provide living cells in the flow channel with nutrients and/or other molecules.

A reservoir comprised in the sample holder may obviate connecting a further reservoir or an aspiration/removal device to the channel outlet, and/or it may prevent contamination and/or loss due to spilling of sample fluid.

A further method step may comprise, prior to forming the first and/or second layer, in particular prior to loading the flow channel with a liquid and first objects, providing at least part of the ceiling with a functionalisation layer and/or providing at least part of a channel wall, in particular at least part of a floor opposite the ceiling, with an adhesion prevention layer.

Such functionalisation layer may promote or reduce binding of the (further) cellular objects to the ceiling. A functionalisation layer may be provided locally. Such a functionalization layer may be a layer that promotes cell adhesion in general or it may be a layer promoting binding only of specific cells (e.g. the target cells). Providing an adhesion prevention layer, or: antifouling layer, allows localising binding of objects, in particular cellular objects, to a surface, which may assist preventing and/or promoting particular interactions, and/or which may assist further use, e.g. promoting that binding only occurs in a region of interest for optical detection and/or for application of a particular force.

Suitable coating agents for attachment of target cells are known in the art. Such coatings preferably comprise a polypeptide. Such a coating may comprise a polypeptide selected from the group consisting of fibronectin, poly-L-lysine, poly-D-lysine, poly-L-ornithine, laminin, collagen, fibronectin, fibrinogen, vitronectin, osteopontin thrombospondin, VEGF, VCAM-1, ICAM. In another further embodiment, the coating comprises one or more from the group consisting of fibronectin, poly-L-lysine, poly-D-lysine, poly-L-ornithine, laminin, collagen, fibronectin, fibrinogen, vitronectin, osteopontin thrombospondin, VEGF, VCAM-1, and ICAM. These polypeptides may attach to the surface and in their turn subsequently attach with the target cells thereby attaching the target cells to the surface. In any case, a suitable coating comprising a polypeptide may be selected such that the target cells that are attached to the surface allow for the target cells to remain attached to the surface when applying a force on the effector cells that are bound thereto. In other words, when a centrifugal force is applied on the effector cells which allows for these cells to detach from the target cells, the target cells are to substantially remain attached to the surface.

Also or alternatively, antifouling properties of the device surface may be achieved either by choosing a polymer that has inherent antifouling properties. Or by applying a physisorbed antifouling layer, e.g. a hydrophobic surface may be coated by applying an aqueous solution containing a PEG block co-polymer e.g. Pluronic-F127 that physisorbes and presents a PEG-brush surface with low biofauling properties. Chemical functionalization may also be achieved by first attaching a chemically reactive linker to the surface. And then solution coating the device with an anti-fouling molecule that reacts with the activated linker molecule. Pattered coating may be achieved by using a photoactivatable linker and using a photo mask and UV illumination to activate the linker only in select regions of the device.

The method may comprise, e.g. as a portion of the further use, the further step of: determining an adhesion of the first cellular objects to the ceiling, and/or, if applicable, of the second cellular objects to the first layer and/or to the ceiling. Adhesion of biological cellular objects to another object, e.g. a surface and/or a layer may be associated with biological processes, in particular cellular avidity. Determining an adhesion, in particular determining an adhesion strength, of the (further) objects therefore provides information on the biological processes.

The step of determining an adhesion may comprise the further step of: applying a controllable force urging at least some of the first objects and/or, if applicable, second objects away from the ceiling. This allows using and possibly quantifying adhesion properties of the respective objects.

Further, the step of determining an adhesion may comprise applying one more of a centrifugal force, an acoustic force, a magnetic force, an optical force, and a shear force to the first and/or, if applicable, second objects.

Suitable methods for applying one or more such forces in controllable manner have been developed, e.g. see WO 2011/153211 regarding use of centrifugal forces, and WO 2018/083193 regarding use of acoustic force.

For any of these forces, either alone or in any suitable combination, providing a first and/or second layer of (first and/or second) objects on the ceiling, or: a “ceiling incubation method”, as provided herein provides a way to improve control over incubation times and/or interaction times. Moreover, after detachment of first and/or second objects from the ceiling, chances of recontaminating remaining objects on the ceiling by detached objects are reduced. Selectivity of methods relying on controlled detachment by controlled application of forces are therefore improved.

Any method herein may comprise the step of removing unbound first cellular objects and/or, if applicable, unbound second cellular objects from the channel, in particular by flushing a liquid through the channel.

Such further step of removing unbound first cellular objects from the channel may comprise removing unbound first cellular objects from the channel between the step of arranging the sample holder in the upright orientation and the step of loading the flow channel with a liquid containing second cellular objects

Thus, unbound cellular objects may be removed for preventing interaction between bound an unbound (first and/or second) cellular objects. Also or alternatively, the unbound first and/or second cellular objects may be removed for further use, e.g. analysis, study, administration to a subject, e.g. Also or alternatively, bound first and/or second cellular objects may be removed for such further use, in particular after removal of the unbound first and/or second cellular objects, the removal possibly comprising non-force-dependent separation methods known in the art.

By the present concepts a “ceiling binding method” and for biological studies a “ceiling incubation method” are provided, simplifying microfluidic studies.

Associated with the foregoing and any benefits discussed, herewith is provided a sample holder having a flow channel extending between a channel inlet at an opening side and a channel outlet at the opening side and defining a ceiling associated with the opening side, wherein at least part of the ceiling is provided with a functionalisation layer and/or with first cellular objects, in particular biological cells such as target cells, attached to the ceiling.

Such sample holder may be used for cellular avidity studies, e.g. for identifying effector cells, such as e.g. immune cells, that target target cells, such as e.g. tumor cells immune cells that target tumor cells. The functionalization layer provided on the ceiling may facilitate and/or improve adhesion of cells (e.g. target cells) to form a cell monolayer in accordance with any method described herein.

Associated with the foregoing and any benefits discussed, herewith is also provided, a sample holder having a flow channel extending between a channel inlet at an opening side and a channel outlet at the opening side, in particular a sample holder as discussed before herein, wherein at least part of a channel wall, in particular at least part of a floor opposite the ceiling, may also be provided with an adhesion prevention layer.

This may facilitate localising binding of an object to a channel wall to binding to the ceiling, e.g. this may prevent binding of objects to surface areas where binding is not preferred.

Associated with the foregoing and any benefits discussed, herewith is also provided, a sample holder having a flow channel extending between a channel inlet at an opening side and a channel outlet at the opening side, in particular a sample holder as discussed before herein, wherein the sample holder comprises a liquid reservoir in fluid communication with the channel outlet, wherein the flow channel defines a channel volume between the channel inlet and the channel outlet for holding a liquid, and the liquid reservoir defines a reservoir volume for holding a liquid of at least one time the channel volume, preferably at least 2 or 3 times the channel volume, for holding in the upright orientation a liquid in the flow channel and the reservoir at a liquid level above the channel inlet and/or channel outlet.

Such sample holder facilitates manipulating liquid samples in the flow channel. E.g., sample liquid in the flow channel can be modified by addition of sample material, in particular liquid, without requiring (simultaneous) removal of sample material. This may simplify studies and it may prevent contamination of surroundings and/or removal devices.

The sample holder may be configured for use in a microscopic method, wherein at least part of the flow channel is configured for microscopic imaging of flow channel contents, e.g. cellular objects and/or layers of objects.

The sample holder may contain in the flow channel a liquid, wherein the liquid and the channel inlet and/or channel outlet are configured to retain the liquid in the flow channel by capillary force when the sample holder is inverted into an upside-down orientation. This may facilitate forming a layer on the ceiling of the flow channel. However, in any case a closure to the channel inlet and/or outlet may be provided, e.g. for (perceived) security and/or to prevent contamination.

Such closure may be or comprise (use of) a cap or plug. Alternatively to closing inlets and outlets with a cap or plug, the inlets and/or outlets may also be closed by other means such as a sealing membrane, film, foil or mat. In an embodiment the sealing membrane, film, foil or mat is pierceable, for example by a syringe, needle or pipette, without losing its sealing capability. In an embodiment, liquids present in the inlets and/or outlets are removed.

The liquid may contain first objects, in particular cellular objects such as biological cells e.g. target cells, and/or it may contain second objects, in particular second cellular objects, more in particular further biological cells which may interact with the first cellular objects, such as effector cells binding to target cells.

In such sample holder, the reservoir may surround the channel outlet. Also or alternatively the channel outlet may be arranged in, and in fluidic communication with, the reservoir, in particular being arranged in a floor of the reservoir. Also or alternatively, the reservoir may have an outlet at the opening side larger than the channel inlet and/or channel outlet; the reservoir opening may be, but need not be, configured to retain sample liquid in the flow channel by capillary force when the sample holder is inverted into an upside-down orientation. A larger opening may one or more of facilitate construction of the sample holder, facilitate adding and/or removing sample liquid, increase liquid surface area for exchange with surrounding gases such as improving oxygenation of the liquid.

A sample holder as described herein may comprise a plurality of flow channels extending between a respective channel inlet at an opening side and a respective channel outlet at the opening side. Further, each flow channel may be provided with a respective liquid reservoir in fluid communication with the respective channel outlet. Preferably, each reservoir is only connected with a single channel.

Such sample holder facilitates multiple studies in parallel. Moreover, the “ceiling incubation method” as described herein, facilitates precise control over contacting/incubation times of objects with the (functionalized) ceiling and/or of second cells with the first layer even in such multi channel sample holders where loading of samples into different channels may be done sequentially. By flipping the sample holder with all channels at once, the objects in all samples/flow channels can be made to contact the respective ceiling and/or any layer thereon at substantially the same time.

### BRIEF DESCRIPTION OF THE DRAWINGS

The above-described aspects will hereafter be more explained with further details and benefits with reference to the drawings showing a number of embodiments by way of example.

FIG. 1 is a photograph of a sample holder, here a microscopy slide provided with a flow channel;

FIG. 2 indicates a sample holder in cross section and steps of a method disclosed herein;

FIG. 3 indicates a sample holder comprising plural channels;

FIGS. 4-5 show arranging plural sample holders for application of a centrifugal force;

FIG. 6 indicates, like FIG. 2, steps of a method disclosed herein;

FIG. 7 indicates cell-cell interaction;

FIGS. 8-10 indicate method steps with another embodiment of a sample holder, in cross section view;

FIGS. 11-15 show variants of a sample holder in cross section and top view, respectively.

### DETAILED DESCRIPTION OF EMBODIMENTS

It is noted that the drawings are schematic, not necessarily to scale and that details that are not required for understanding the present invention may have been omitted. Further, elements that are at least substantially identical or that perform an at least substantially identical function are denoted by the same numeral, where helpful individualised with alphabetic suffixes.

Further, unless otherwise specified, terms like “detachable” and “removably connected” are intended to mean that respective parts may be disconnected essentially without damage or destruction of either part, e.g. excluding structures in which the parts are integral (e.g. welded or moulded as one piece), but including structures in which parts are attached by or as mated connectors, fasteners, releasable self-fastening features, etc. The verb “to facilitate” is intended to mean “to make easier and/or less complicated”, rather than “to enable”.

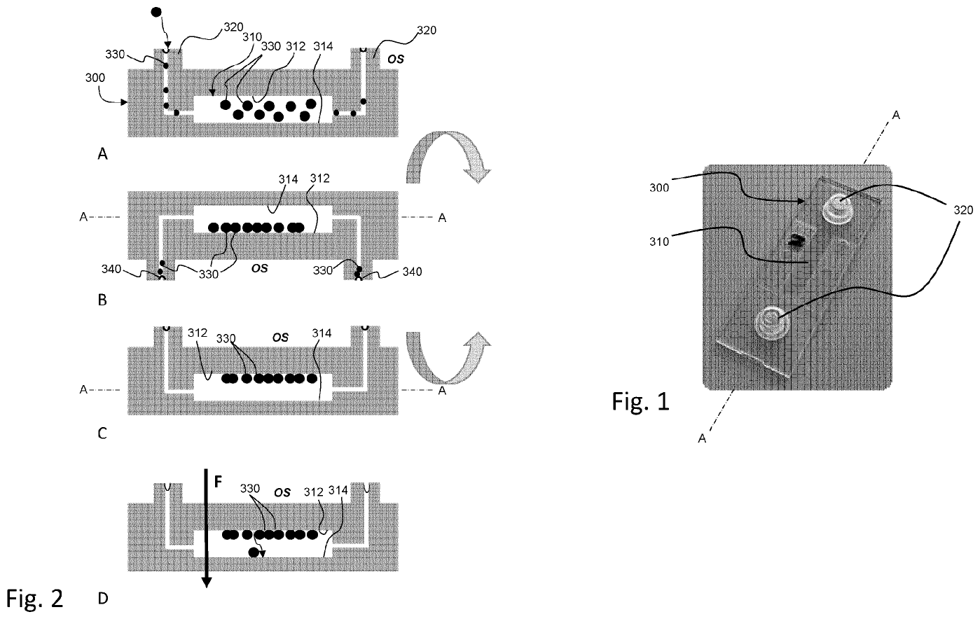

FIG. 1 is a photograph of a channel slide used as a sample holder 300 in centrifuge experiments. Indicated are the position of a main channel 310 and a channel inlet and a channel outlet 320 of the channel slide. The inlet and outlet 320 may be identical; identification as “inlet” or “outlet” may be determined only in use when adding or removing sample material into the channel 310, whereas from the slide itself no difference is discernible. The shown slide is of a well-known format in the field of cell culturing, well known in the field of cell culturing. As an option, the shown inlet and outlet 320 are provided with Luer connectors.

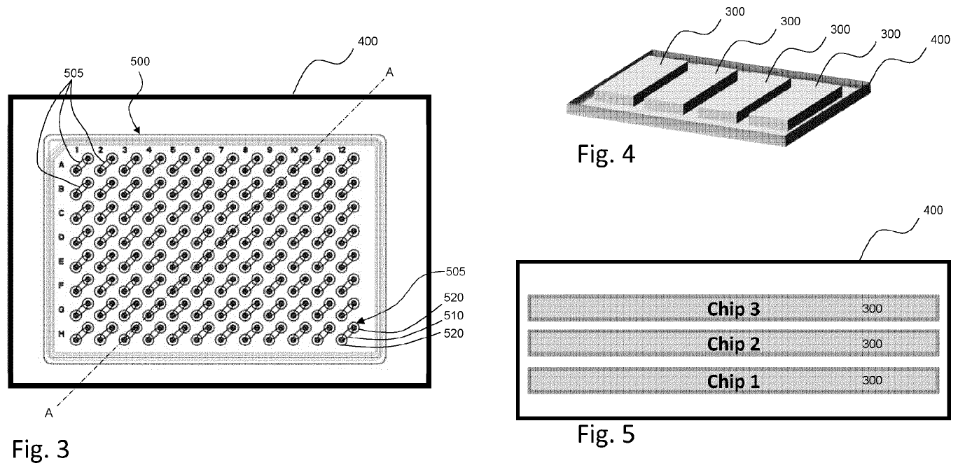

FIG. 2 schematically describes a method for performing centrifuge experiments using a sample holder 300 e.g. a channel slide. FIG. 2 schematically shows the sample holder 300 in cross section, having a flow channel 310 extending between a channel inlet 320 and a channel outlet 320 at an opening side and defining a ceiling 312 and a floor 314 associated with the opening side OS. In an upright orientation of the sample holder the opening side OS is upward. Thus, the channel can be loaded via the inlet 320 from above.

Shown is in step A the loading liquid of cells 330 in a liquid into the channel 310 via channel inlet 320. In particular, in the upright orientation of the sample holder 300 which the opening side OS is upward, the channel 310 is loaded with a liquid containing the cells 300.

In step B the channel slide 300 is flipped upside down to allow the cells 330 to sediment to the “ceiling” of the channel (which is optionally coated with a functionalisation layer and/or target cells as described herein). The channel inlet 320 and outlet 320 are left open during the inverting (flipping). The liquid, the inlet 320 and the outlet 320 are chosen so that the liquid is maintained in the flow channel by capillary force. The inverting is preferably along an axis A of the holder 300 which is selected to be along a main direction of the channel 310 and/or parallel to an axis between inlets 320 and outlets 320. This way, unwanted fluid flow in the channel 310 may be further minimized and/or avoided further during handling of the sample holder 300 (see axis A and arrow).

Some of the cells 330 sediment into the channel inlet 320 and outlet 320 but the cells 330 and fluid are trapped inside the channel slide due to capillary force as indicated by the fluid meniscus 340. The channel slide is kept in the inverted upside-down orientation for a defined period for incubation. Thus a layer of the cells 330 on the ceiling is formed.

In step C the channel slide 300 is flipped upright with the cells now “hanging” from the ceiling (the top side of the fluid channel). Unbound cells 330 may be rinsed from the channel 310, e.g. by flowing liquid through the channel. The channel slide 300 may then optionally be placed in a system for application of a force (see step D).

In step D a force such as a centrifugal force is applied indicated with the black arrow labelled F and cells may detach from the target cells and/or channel wall as indicated here for a single cell. Any detached cells may be removed from the channel and subsequently be used for further study and/or application. The cells remaining bound may thereafter be removed from the channel and subsequently be used for further study and/or application. Also or alternatively, at least some of the cells may be left on the ceiling and be used for further study and/or application as set out below. It is noted that the channel inlet and outlet are oriented opposite to the direction of the force F, thus sample liquid is retained. Centrifugal force may assist in removing gas (air) bubbles from the sample liquid.

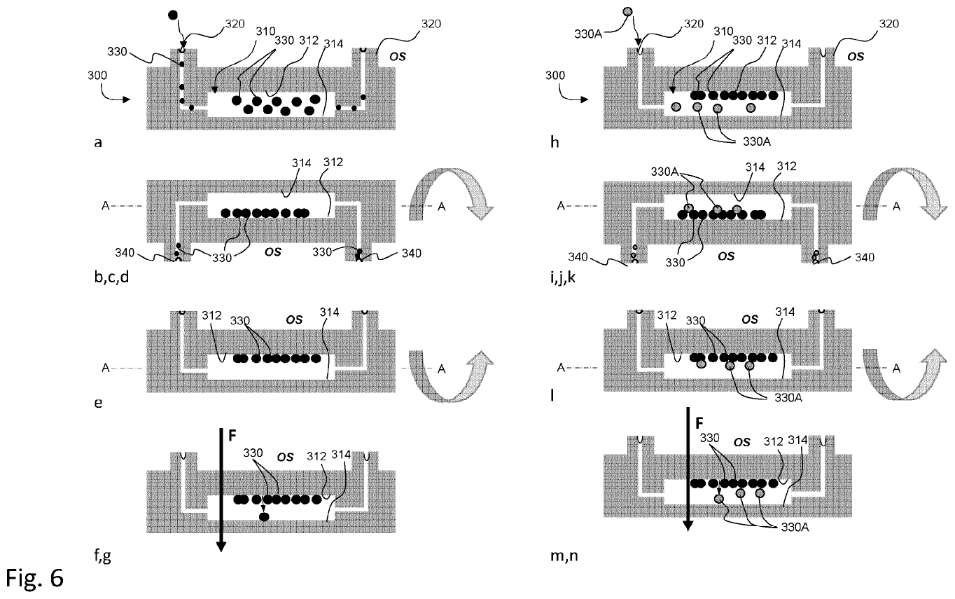

FIG. 3 shows in top view, a sample holder 500 comprising a plurality of units 505 each comprising a flow channel 510 extending between a respective channel inlet 520 and a respective channel outlet 520 at an opening side. The sample holder 500 allows parallel processing of multiple samples according to the presently presented concepts. The units 505 and flow cells 510 therein may be arranged in any suitable pattern and/or number, e.g. in accordance with a layout that can be used by a standard pipetting robot; here, the sample holder 500 conforms to the layout of a 96 wells well plate and comprises 48 channels. Here, the channels 510 are oriented diagonal to the rectangular shape of the holder 500. In another embodiment, it may be convenient to orient the channels along a natural axis of the sample holder, e.g. parallel to circumference of a multi well plate, e.g. rectangular sides. This may facilitate a natural rotation axis for the inversion.

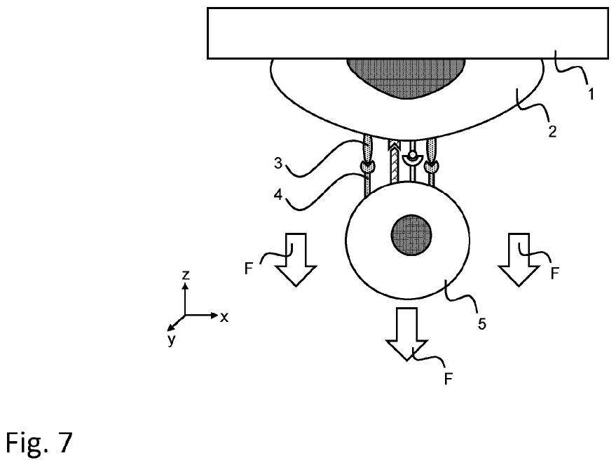

FIGS. 4-5 show that several channel slides 300 or 500 (any of which may be referred to as “chip”) may be subjected to centrifugal force simultaneously by placing the respective sample holders side by side (FIG. 4) and/or superposed (FIG. 5) in a centrifugal bucket 400 which may be custom made. Note that other arrangements and/or other forces may also be used.

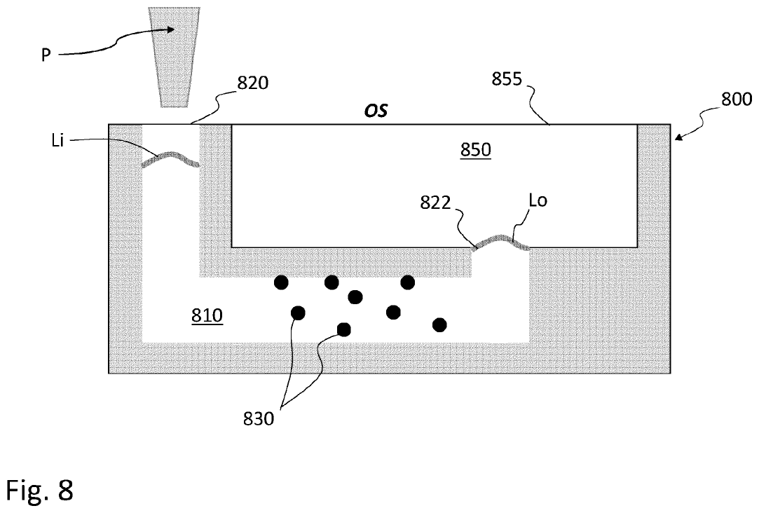

FIG. 6 schematically describes another method for performing centrifuge experiments using a sample holder having a flow channel 310 extending between a channel inlet 320 and a channel outlet 320 at the opening side and defining a ceiling 312 associated with the opening side OS.

Just as in FIG. 2, in step a, in the upright orientation of the sample holder in which the opening side OS is upward, the flow channel is loaded via the channel inlet 320 with a liquid containing first cellular objects, in particular biological cells such as target cells.

Steps b-d comprise forming a layer of the first cellular objects on the ceiling 312, comprising inverting the sample holder 100 into an upside-down orientation, while maintaining the inlet an outlet open, allowing sedimentation of the cellular objects onto the ceiling 312, and incubating the first cellular objects for a determined defined period in the upside-down orientation to promote binding of least some of the cellular objects to the ceiling.

Step e comprises arranging, after the incubation, the sample holder in the upright orientation for a further use, for example rinsing unbound cells from the channel and/or placing/inserting the sample holder in a centrifuge holder.

Steps f-g comprise applying a controllable force urging at least some of the first cellular objects away from the ceiling and removing unbound first cellular objects from the channel.

Steps h-n generally repeat method steps a-g to form a second layer of second cells 330A onto the first layer of cells 330 and removing unbound cells 330, 330A from the channel.

At least part of the method may be done automated, for instance, using a multi-channel well plate as in FIG. 3, the steps indicated in FIG. 6 may be performed as follows:
a. A pipette robot capable of inflow and outflow with multi tips fills the channels 310, 510, in the upright orientation of the sample holder, with desired cells such as target cells 330, e.g. tumor cells. The pipette robot may pipette different kinds of cells into different channels.
b. Inversion of the well plate: a human operator or a flipping robot then turns the chips upside down (an operator may do so manually and/or using a flipping tool). Although a closure may be used on the channel inlet or outlet, by appropriate selection of the liquid, channel inlet and channel outlet, capillary forces and surface tension of the liquid may prevent leakage and obviate a closure. Luer connectors at 6 mm diameter opening or less, e.g. 5.5 mm, 5 mm, 4.5 mm, 4 mm, 3, 5 mm or 3 mm or less diameter tend not to require closures for aqueous liquids such as cell culturing solutions.
   The inversion is preferably along an axis A of the holder 300, 500 which is selected to be along a main direction of the channels 310, 510 and/or parallel to an axis between inlets 320, 520 and outlets 320, 520 of the channels to minimize and/or avoid unwanted fluid flow in the channels 310, 510.
c. The cells sediment on the ceiling of the microfluidic channel in the flipped (upside down) orientation.
d. Incubation, possibly in an incubator, for a defined period to promote attachment of a monolayer of the cells.
e. Rearranging of the channel plate: a human operator or a flipping robot reverts the chip to standard upright orientation. The cells 330 remain attached to the ceiling 312. Thus, a layer of cells 330 is formed on the ceiling 312 of the channel 310 and the cells 330 now hang from the ceiling.
   Optionally, a pipette robot capable of inflow and outflow, possibly with multiple tips, replaces the media (sample liquid(s)) and removes the excess of cells that did not adhere to the surface.
   Further, the channel plate is mounted for application of a force, here centrifugal force.
f. Application of a controllable force F. E.g. for centrifugal force the plate is spun at a desired G-force to detach unbound/loosely bound monolayer cells.
g. A pipette robot capable of inflow and outflow, possibly with multiple tips, replaces the media (sample liquid) and removes the excess of cells that did not adhere to the surface.
h. Effector cells 330A or other cells are introduced with the pipette robot into the channels 310.
i. Inversion of the well plate: a human operator or a flipping robot then turns the chips upside down. The channel inlet and outlet may be closed or, preferably, may remain open again, relying again on capillary forces.
j. The effector cells 330A sediment on the ceiling 312 of the microfluidic channel 310 and the first layer of cells 330 formed thereon in the flipped (upside down) orientation.
k. Incubation for a defined period of effector cells 330A on the ceiling 312 and the first layer of cells 330 on it.
l. Rearranging of the channel plate to standard orientation (“flipping back”). The effector cells 330A remain attached to the cells 330 on the ceiling 312. Thus, a second layer of cells 330A is formed on the ceiling 312 of the channel 310 and the cells 330 and 330A now hang from the ceiling 312.
   Optionally, a pipette robot capable of inflow and outflow, possibly with multiple tips, replaces the media (sample liquid) and removes the excess of cells that did not adhere to the surface.
   Further, the channel plate is mounted for application of a force, e.g. centrifugal force.
m. Application of a controllable force F. E.g. the chip is placed in a centrifuge and rotated to apply a defined multiple of Gs (1 G=1 times gravity)
n. A pipette robot capable of inflow and outflow, possibly with multiple tips, replaces the media and collects the media with the desired detached cells 330A

The method may optionally comprise application of one or more further layers, e.g. by one or more repetitions of steps h and further, with further objects such as one or more further types of cells. Note that any pipetting may also be done by a human operator in addition to or instead of (use of) a pipetting robot. Also, replacement of media may be left out.

After and/or during formation of the first and/or second layer (and/or any subsequent layer) and/or after application of a force, at least part of the formed layer may be imaged and/or otherwise studied. E.g. Imaging of effector cells on the first layer may be done to determine a fraction of attached and detached cells, which may be used as a measure of cell avidity.

Also or alternatively, selection of cells based on attachment/detachment force, and/or quantifying detachment force for some cells may comprise repetition of steps l+m or steps l+m+n.

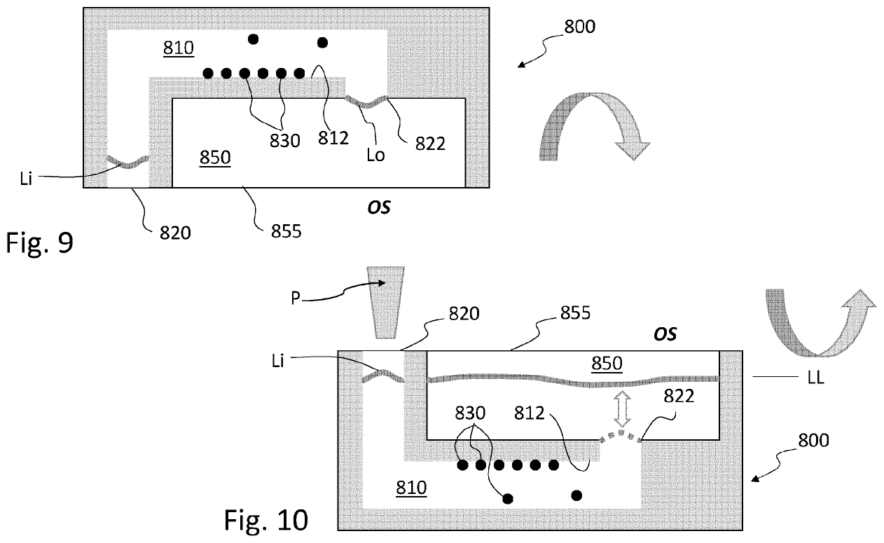

FIG. 7 schematically indicates a target cell 2 and an effector cell 5 (here being a cell of interest) and cellular avidity. Target cells 2 are provided on a surface 1, in particular a ceiling 312 of a channel 310 which as depicted in this case is a flat surface. The target cell 2 expresses ligands and receptors 3, likewise the effector cell 5 which is to be targeting the target expresses ligands and receptors 4 as well. A specific ligand-receptor interaction (3 and 4) can be the driving force for the forming of a cell-cell bond with multiple ligand-receptor interactions combined resulting in strong cell-cell binding. To rupture this cell-cell bond, a force F is exerted on the effector cell 5 away from the target cell 2, which can be along the z-axis direction e.g. when the flat surface is defined as being in the x-y plane. Alternatively, this can also be in the x or y-axis direction, e.g. a shear force due to liquid flow along the surface. When the cell-cell bond is ruptured, the effector cell 5 moves away from the surface 1 and/or the target cell 2 and this event or the amount of cells bound, can be detected by imaging techniques (microscopy, video, etc.) and/or detached cells can be collected and then quantified and/or further analyzed.

It should be noted that the present concepts also facilitate studying aspecific interactions e.g. such as when control cells (without a specific antigen receptor) are used or when cells bind to surfaces such as glass or PLL/fibronecting etc. which may have been used as an adhesion promoter and/or as part of another functionalisation layer. Determining cellular avidity for such interactions may also be of interest.

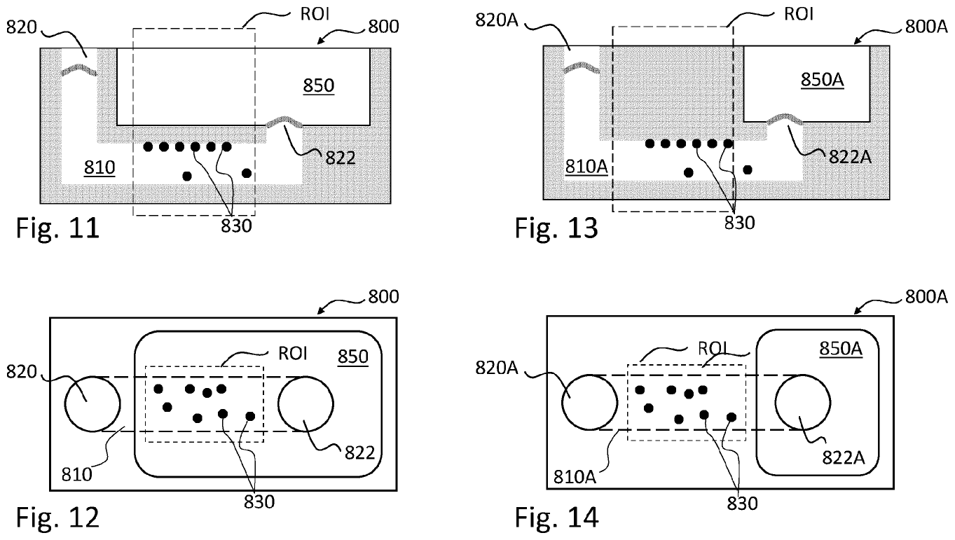

FIGS. 8-10 show another sample holder 800, or flow cell, which may be used in any method disclosed herein. The sample holder 800 has a flow channel 810 extending between a channel inlet 820 and a channel outlet 822 at an opening side OS. The sample holder 800 further comprises a liquid reservoir 850 surrounding and being in fluid communication with the channel outlet 822 and therewith in fluid communication with the channel 810. The reservoir 850 has, as an option, an opening 855 significantly wider than that of the channel inlet 820 and/or outlet 822. Note that, again, the words “inlet” and “outlet” may refer to a situation in use and are mainly used in the following for ease of reference and associated with a preferred manner of use.

The flow channel 810 defines a channel volume V_{C} between the channel inlet 820 and the channel outlet 822 for holding a liquid, and the liquid reservoir 850 defines a reservoir volume V_{R} for holding a liquid of at least one time the channel volume V_{C} (V_{R}=nᵥ×V_{C}, nᵥ>1, preferably nᵥ>2 or even nᵥ>3). The channel volume V_{C} may typically be a few tens to a few hundreds of microliters. The reservoir volume V_{R} may be few to several hundreds of microliters or even up to a few millilitres.

Like the sample holders discussed before, the channel 810 may be loaded with a sample liquid containing objects, such as cellular objects, 830. The loading may be done from above in an upright orientation, e.g. using a pipette P (FIG. 8) or a syringe, possibly provided with a needle. The channel 810 is preferably loaded to a liquid level up to a boundary of the channel outlet 822, although in the channel inlet 820 the liquid level Li may be higher, since capillary forces may retain the liquid in the channel outlet level Lo. A layer of objects 830 may be formed on the ceiling 812 of the channel by inverting the sample holder 800 into an upside-down orientation (FIG. 9). By appropriate selection of sample holder material, size of the inlet and outlet and sample liquid, in the upside-down orientation the liquid and any objects retained therein may be held in the channel by capillary force.

After reverting the sample holder 810, the channel 810 further liquid may be added to the sample holder 800. The further liquid may be added via the inlet 820, e.g. using a pipette P, for flushing the channel 810 wherein liquid is displaced, possibly including unbound objects 830, into the reservoir 850 (FIG. 10—see arrow). The liquid can be easily removed from the reservoir in a separate operation from the addition, and/or at least part of the liquid can be left in the reservoir 850, for holding in the upright orientation a liquid in the flow channel 810 and the reservoir 850 at a liquid level LL above the channel outlet 822. The thus retained extra volume of liquid in the reservoir 850 allows for one or more of improving stable cell culture conditions, reduction of osmotic shifts with evaporation, and chances or drying out of the channel 810. Note that the outlet 822 may be quite short, here only the thickness of the ceiling wall 812, reducing diffusion length for oxygen to the channel.

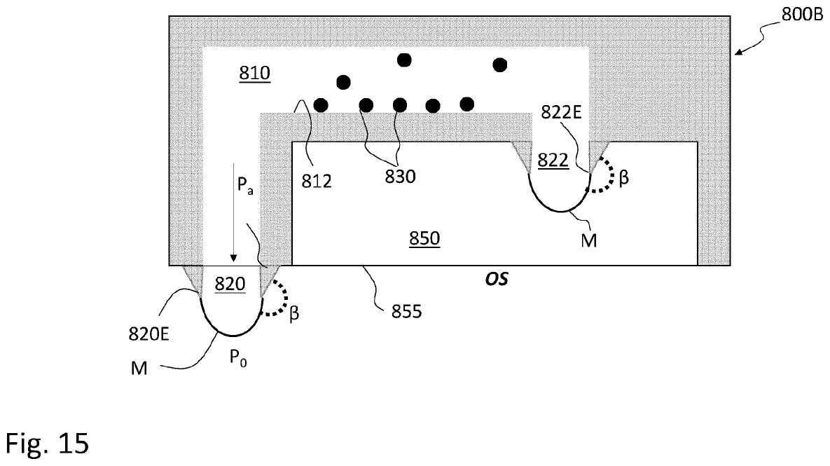

FIGS. 11-12 indicate a sample holder 800 in cross section view and top view, respectively, and FIGS. 13-14 likewise indicate a variant 800A of the sample holder 800. In each figure a region of interest ROI for optical assessment of (contents of) the channel 810, 810A, is indicated. The two variants 800, 800A differ predominantly in the size and location of the respective reservoirs 850, 850A; in FIGS. 11-12 the reservoir 850 is relatively large and overlaps the region of interest ROI, whereas in FIGS. 13-14 the reservoir 850 is relatively small and is arranged aside the region of interest ROI. The latter may reduce affecting the optical access by sample liquid and possibly any objects therein in the reservoir 850. Any corners in the reservoir and/or the channel may be rounded for simplifying emptying and/or cleaning.

Such sample holder may also be used for microfluidic experiments and/or cell culturing experiments not using inversion and/or cellular adhesion to the ceiling of the respective channel. Also or alternatively, in a multi-channel sample holder, e.g. of the type shown in FIG. 3, one or more units, possibly each unit may be provided with a reservoir like sample holder 800/800A.

In some sample holders provided herein, e.g. as shown in FIG. 15 for a variant of the sample holder of FIGS. 8-14, the inlet and/or outlet may be provided with a sharp edge 820E, 822E. This may improve liquid retention and help acting as a passive stop-valve. E.g. this may increase capillary forces by pinning a liquid meniscus M and/or it may assist preventing reducing liquid loss in an inverted orientation. The increase in angle μ between the surface of the inlet/outlet adds to the surface contact angle (θa) which may effectively neutralize a difference Δp between the atmospheric pressure P₀ and gravity and capillarity induced pressure Pₐ as indicated in FIG. 15.

Associated with the above description of method steps and sample holders, it is understood that “cellular avidity” as used throughout herein comprises the overall strength of interactions occurring in a cell to cell contact and/or a cell to surface contact (coated surface or not), involving a diversity of molecules at the surface(s) of the cell(s) that interact(s). Such interactions may include a diversity of receptor-ligand pairs, among which e.g. a specific receptor-ligand interaction, occurring at the membrane surface of a cell. For example, when a T-cell receptor triggers the formation of an immune synapse by recognizing an antigen presented by an MHC molecule at an antigen presenting cell, the synapse formation involves such multitude of interactions, as also other membrane bound molecules are involved in the interactions (such as integrins and the like). Hence, “cellular avidity” may not be restricted to the interaction of e.g. the alpha and beta chain of the T-cell receptor and the antigen presented by MHC, but rather involves a multitude of interactions working jointly forming a strong bond between e.g. cells. It may also involve active signalling and processes internal to the cells such as e.g. during immune synapse formation. It is understood that the cellular avidity of a cell of a certain type is defined relative to its target cell and conditions tested

The term ligand and receptor in accordance with the invention may define their inter-relationship. The term receptor may not be construed to be limiting in any way and is understood to mean a protein presented at the cell surface which can (specifically) interact with another protein (ligand) presented at another cell. The terms ligand and receptor are used to indicate a complementarity which is important for specific recognition between cells without restrictions on the complementary molecules that can be contemplated.

In some method steps the target cells are provided attached on a surface, in particular the ceiling of the channel. Providing the target cells attached to a surface as such is well known in the art. For example, a glass or plastic surface may be utilized to attach cells thereto. A surface material may be preferred which allows for detection of attachment to and/or detachment from the target cells, i.e. cells carrying a receptor. Such a surface material for instance also may allow for microscopy methods (e.g. by being a transparent material). In order to attach cells to the surface, the ceiling surface may be pre-treated with a coating such as a polypeptide. Suitable polypeptides include e.g. poly-L-lysine or the like. Suitable polypeptides for attachment of target cells that may be contemplated and are known in the art include for example fibronectin, poly-L-lysine, poly-D-lysine, poly-L-ornithine, laminin, collagen, fibronectin, fibrinogen, vitronectin, or osteopontin. In any case, a suitable polypeptide or other suitable coating if needed may be selected such that the target cells that are to be attached to the surface are promoted to attach to the surface and allow for the target cells to remain attached to the surface when applying a force on the cells carrying the receptor. In other words, when a force is applied on cells of interest which allows for these cells to detach from the target cells, the target cells may substantially remain attached to the surface, dependent on the force. Thus, cells may be distinguished based on their attachment force.

In another embodiment, instead of providing target cells attached on a surface, a functionalized wall can be provided which provides the cells with a receptor a suitable surface to attach to in the means and methods in accordance with the invention as described herein throughout. A functionalized wall in accordance with the invention presents ligands/receptors in a similar fashion as they are presented on a cell surface, e.g. in a lipid bilayer, or the like. A functionalized wall thus preferably is functionally equivalent to target cells attached to a surface, and mimics target cells attached to a surface.

In an alternative embodiment, cells with the receptors having different expression levels, when provided separately, may be provided attached to different surfaces as different target cells, and, subsequently the cells previously used as target cells, e.g. a cancer cell (in other embodiments), may be allowed to interact therewith. Cells that have detached and/or remained attached can be subsequently determined and cellular avidity scores provided for each of the cells with different expression levels, and cellular avidity scores can be likewise compared.

E.g. referring to FIG. 6, In steps i-k) the effector cells comprise cells with different expression levels of the receptor contacted with the target cells attached to a surface allow these cells to interact with the target cells. Hence, in this step, the effector cells are introduced on the target cells, e.g. by layering the effector cells thereon. It is understood that this step of contacting is well controlled. For example, as shown in the example section, the step of contacting may be a defined period of 5 minutes. Of course, the step of contacting may be shorter or longer, for example in the range of about 1 minute to about 15 minutes.

Subsequently, e.g. see FIG. 6, step m) a force may be exerted on the effector cells, the force may be in a direction away from the target cells. It is understood that then, the force applied may be perpendicular (in the direction of z-axis) to the surface (x,y) to which the target cells are attached, for example when a centrifugal force or acoustic force is applied. The force may also be lateral (x-axis or y-axis), for example when a shear force is applied such as by flowing a liquid through the flow channel. In any case, the applied force may be controlled such that a defined force is exerted on effector cells carrying the receptor that interacted with the target cells (and formed a bond or not). It is understood that the force that is exerted on the effector cells attached to the target cells is preferably to be substantially equal for all cells, such can be achieved e.g. when using a flat surface. The force required to move a cell away from the target cell preferably can be detected, e.g. via microscopy or other means, to which may be referred to as a cell detachment event. This way, cell detachment events can be monitored and counted.

The number of each of the effector cells with different expression levels of the receptor that have detached from and/or remain attached to the target cells attached to the surface may be determined (e.g. FIG. 6, step n), and using the determined number, in a subsequent step cellular avidity scores may be assigned to each of the cells with different expression levels. By knowing the number of cells that have interacted with the target cells, and knowing the number of cells that remain attached to the target cells, a percentage of each of cells with different expression levels of the receptor that remain attached to the target cells can be determined.

The percentage of effector cells from the contacting step that remain attached to the target cells can be well determined. This can be done by relatively simple means known to the person skilled in the art, e.g. by simply providing a defined number of effector cells to interact with the target cells and, after incubation and applying the force, subsequently collecting detached cells and determining the amount of cells collected, and calculating the percentage that remains bound therefrom. Of course, it may be advantageous and convenient to use microscopy, with which attached cells can be identified and quantified and detachment can be likewise monitored and quantified. As shown in the art, the z-Movi® device offered for sale by the present applicant and its affiliates, applying an acoustic force is well equipped to do so (see also WO 2018/083193. Likewise, similar devices may be provided with microscopy or other means to quantify cells, and attachment and/or detachment events, and also utilizing e.g. shear-force or centrifugal forces instead of acoustic force). Anyhow, at least based on determined numbers of each of the cells with different expression levels of the receptor (that have detached and/or remain attached) a cellular avidity score for the each of cells with different expression levels of the receptor can be determined.

With regard to the cellular avidity score, it is understood that this is to express the strength of binding of cells carrying a receptor to the target cells. It is understood that where we refer to specific forces applied to cells this may refer to average forces, e.g. such forces may not be fully homogeneous, for example over the contact surface as may be the case with acoustic forces and shear-flow forces (See e.g. Nguyen, A., Brandt, M., Muenker, T. M., & Betz, T. (2021). Lab on a Chip, 21(10), 1929-1947) for a description of force inhomogeneities in acoustic force application)

Also for shear-flow forces, the forces may also not be fully homogeneous, for example since the flow speed near the side walls of a flow channel (e.g. with a rectangular cross section) may be lower than in the center of the flow cell (due to the no-slip boundary condition). By choosing a cross section with a high aspect ratio (low and wide) these flow effects may be minimized such that only a few percent of the cells experience a substantially smaller force than the cells in the center of the flow cell. Other methods to mitigate such effects and to specifically select cells that have experienced similar forces may include using flow cell geometries with multiple fluid inlets and/or outlets such that the properties of laminar flow can be used to ensure cells of interest only land in regions of homogeneous force and/or are only selected from regions of homogeneous force. In one example, by using three channel inlets side by side one can use the side channels as sheath flow channels to focus cells of interest inserted into the center channel where the acoustic and/or shear force may be substantially homogeneous. The sheath flow fluid may be the same buffer fluid as is used for the sample cells but then free of sample cells. By increasing the flow speed through the sheath flow channels the cells are more focused and confined to the center of the channel while by reducing the sheath flow speed the cells are allowed to spread out more. Similarly, on the collection side flows in three side-by-side collection channels may be controlled to possibly discard cells flowing close to the channel boundaries and only collecting cells from the center of the channel. By controlling the relative flow speeds of such side channels and the center channel asymmetrically the location of the effective interaction region of the sorting device can be further controlled and cells that have underwent defined forces can be selected and/or detected.

Further means to enable collection of cells from a specific interaction region (and therefore collection of cells that experienced a defined force) include means and methods wherein cells of interest may be provided with a photoactivatable label which may be subsequently activated by illumination with light of a suitable wavelength only in a well-defined interaction region of the device (e.g. near the center of a flow channel or in a center region under an (acoustic) force transducer) to photoactivate and/or switch the dye.

Subsequently the cells can be sorted and counted for example using fluorescence activated cell sorting (FACS) and only those cells which are activated are further used according to the methods described herein thereby obtaining the cells on which defined forces have been exerted. This may for example be highly useful for collecting cells that remained attached to the target cells. For example, the target cells and cells bound thereto may be trypsinized thereby obtaining both the target cells and cells that remained bound thereto in a suspension. Alternatively, attached cells can also be simply collected with physical means (e.g. scraping) from the area of interest, i.e. the surface area with a well-defined nominal force.

For centrifuge forces it is easier to ensure that the force applied is homogeneous across the whole interaction region since such a force does not depend strongly on a location on a surface with respect to a force transducer and/or the wall of a flow channel or sample holder.

Accordingly, in connection to the subject matter disclosed herein, means and methods exist which allow one to exert forces on cells attached to a surface and collect the cells, detached and/or attached cells, on which defined forces have been exerted and determine i.a. the amount thereof, i.e. number of cells.

As the percentage of cells that remains bound at a certain applied force is indicative of cellular avidity, i.e. the larger the percentage of cells that is bound the higher the cellular avidity, it is useful to refer to such a percentage as a cellular avidity score. Of course, one may use a different measure which relates to cellular avidity. One may also refer to the percentage of detached cells instead, wherein conversely a low number is indicative of a relative higher cellular avidity. Instead of percentage, one may also provide the ratio of cells that remain attached divided by the total number of cells that interacted, or provide the ratio for detached cells.

In addition to providing control over cellular avidity by modulating receptor expression, as shown in the example section, cells having the same receptor but varying in cellular avidity in cell population may be advantageously enriched based on their expression level by using force separation methods. This way, the fraction of a subpopulation comprised in the cell population, having e.g. low or high expression, may be enriched for (or, conversely reduced). Hence, in one embodiment, a method is provided for enriching a particular cell population, comprised in a population of cells comprising cells with different expression levels of a receptor, said particular cell population having a relatively higher or lower expression level of the receptor; comprising the steps of:
providing target cells attached to a surface;
contacting a population of effector cells with the target cells attached to the surface to allow the population of effector cells to interact with the target cells;
exerting a force on the population of effector cells;
collecting cells from the cell population which detached from and/or remained attached to the target cells attached to the surface, therewith providing fractions comprising enriched cell populations comprising a relatively higher or lower expression level of the receptor.

Hence, by exerting a force, effector cells having different expression levels of receptors due to e.g. copy numbers, can provide for different cellular avidities and based on this difference, effector cells may detach from and/or remain attached to target cells at different rates at defined forces. By e.g. repeating the enrichment process, highly enriched cell fractions can be advantageously obtained. This may be for example highly advantageous in a scenario wherein a cell fraction is to be prepared for administration to a subject, e.g. a patient. This way, e.g. cells having e.g. predominantly a single copy can be obtained, which may be advantageous when e.g. higher copy numbers are associated with reduced efficacy, exhaustion or side effects.

Enriched cell population thus obtained accordingly with means and methods in accordance with the invention may subsequently in a final step be admixed with a pharmaceutically acceptable buffer or otherwise pharmaceutical acceptably formulated.

In one embodiment, the enriched fraction thus obtained is enriched with cells having a copy number of 1. Of course, suitable means and methods may be selected to provide for optimal enrichment, e.g. of other fractions being enriched for another copy number e.g. 2 and/or related to different expression levels or ranges of expression levels.

Any suitable force application method may be contemplated in accordance with the invention. Increasing the force can be well controlled with acceleration-based methods of applying force such as centrifugation, with shear flow and with acoustic force, which are all suitable means to be used in the methods in accordance with the invention but any other means of controllably causing a force on the cells attached to the target cells thereby forcing them away from the target cells or the functionalized wall surface, may be contemplated. In any case in any of the methods as described and as outlined herein, and as described above, the applied force may be an acoustic force, a shear flow force or an acceleration force such as a centrifugal force. The latter may provide a homogenous force distribution over a channel or plurality of channels, and may be preferred. In a further embodiment, embodiment, the applied force in the means and methods of the invention is a force ramp, preferably a linear force ramp. It is understood that the different forces that are to be applied can be constant forces applied for a defined period. The forces applied may be in various forms as a function of time. Preferably however, the applied force is an increasing force, that is, after the incubation step, an increasing force is applied for a defined period until a defined end force is reached. For example, with e.g. an acoustic force in about 150 seconds, a defined end force is reached of 1000 pN. Increasing the force is preferably done in a linear force ramp, but other ways to increase the force over time may also be used (e.g. exponential loading where the force is doubled over a certain time period and keeps doubling until a defined end force is reached). Hence, in a preferred embodiment the applied force is a force ramp, preferably a linear force ramp.

As said, any suitable material may be selected for attaching the target cells. Preferably, the surface may be glass or plastic, as such materials are known to be highly suitable for attaching cells and may allow for visual inspection of the attached cells and cells carrying a receptor interacting therewith. The target cells that are attached to the surface, preferably are attached as a monolayer. The monolayer preferably is at high confluency. The subsequent cells that are to interact with the target cells are preferably provided in a relatively low cell density as compared with the target cells, such that substantially all cells that are to interact with target cells are outnumbered (there are more target cells). Such provides for advantageous controllable conditions when applying the force on the cells that interact with the target cells.

In a preferred embodiment the height of a well or flow channel is from 200 to 400 μm. This height is optimal because the volume to area ratio is sufficient to allow for cells to create a monolayer and not run out of nutrients too fast. A height from 200 to 400 μm generally leads to a cell settling time of from 2.5 and 4.5 minutes. Increasing the height would mean increasing the cell settling time which is unwanted, as the higher the cell settling time is, the higher the heterogeneity in cell setting time becomes. For example, effector cells when flushed in will be distributed at multiple heights throughout the well or channel. Cells starting from a smaller height will take less time to sink down to the target cell monolayer and thus take less time to settle than cells starting from a larger height. This difference becomes larger, the larger the channel height.

The concentration of the effector cells may depend on many factors including the height of the well or flow channel used, for example the concentration of effector cells may be 0.5-1 e6/ml for a height of 400 μm and 1-2e6/ml for a height of 200 μm. The indicated concentrations are optimal for the indicated heights as it provides cell numbers that enable to distinguish single cells with imaging software used in the art and at the same time to have good data quality.

### Examples—Materials and Methods

### Effector Cell Preparation

Untransduced or FMC63-transduced primary CAR T cells were obtained from ProMab Biotechnologies Inc. (Anti-CD19-TF-CD28-CD3z (PMC-152)). Two days before the avidity experiments primary T cells (untransduced (UNT) and CAR transduced) were thawed and purified. UNT and CAR T cells were derived from the same donor. After thawing, a fraction of the CAR transduced T-cells was purified using magnetic activated cell sorting (MACSBeads, Miltenyi) using an anti-flag antibody. The concentration of antibody was titrated to enrich for the T cells with the highest CAR expression. This was the CAR High T-cell population. The flow-through was also collected. This was the CAR Low T-cell population. The four populations: UNT, CAR unpurified, CAR High and CAR Low T-cells were put in culture with interleukin 2 (IL2) supplement for two days in preparation for the avidity experiment. These were used as the effector cells.

### FACS Analysis

The effective transduction of the cells and the MACS purification procedure to obtain samples with low and high expression levels of the CAR receptor were checked using standard FACS analysis. The relevant FACS plots are shown in FIG. 1. When looking in the FLAG-PE PE-A channel it is clear that the transduced CAR-T population (CAR unpurified) contained (almost) no untransduced cells but did contain at least two distinct populations with lower and higher expression levels of the CAR (seen as two peaks in the graph). The MACS separation resulted in two distinct fractions: one fraction with (almost) purely the low expression level (CAR low) and another fraction with predominantly the higher expression level (CAR high).

### Target Cell Monolayer Preparation

z-Movi® chips, channel slides (ibidi, #80166) and 18 mm circular glass slides were treated with NaOH 1 M for 1 h, rinsed with water and let dry in a dry 37° C. incubator. Subsequently, after 1 h, the chips and slides were coated with Poly-L-Lysine (#P4707-50ML, Sigma) diluted 1:5 in PBS for 10 minutes at room temperature and left to dry overnight in a dry 37° C. incubator. The following day Nalm6 (CD19+) or HeLaCD19+ cells were used for seeding monolayers of target cells. For the 18 mm circular glass slides, a 4 well silicone insert (ibidi, #80489) was stuck to the PLL-treated glass surface prior to seeding. For both Nalm6 and HeLa cell lines, cells were counted and seeded in serum-free medium on the different vessels at a concentration required to achieve a monolayer confluence close to 100%. For the channel slides the monolayers were seeded on the ceiling of the channel (see FIGS. 2 and 6). 60 μl of the target cell suspension was pipetted into the inlet and seeded by gravity by tilting the slide for few seconds until the fluid reached the outlet. 60 μL is the volume of the channel, so this way the whole channel is filled, and air plugs in inlet and outlet keep the liquid from moving. The slide was incubated upside down in a humidity and CO₂ controlled incubator for 30 minutes. Medium was exchanged by applying 60 μl of new medium to the inlet and withdrawing 60 μl from the outlet, 4 times. The slide was put back upside down in a humidity and CO₂ controlled incubator.

The vessels were placed for 30 minutes either in a dry incubator (z-Movi® chips) or in a humidity and CO₂ controlled incubator (channel slides and circular glass slides), then the medium was exchanged with a serum-containing one. The chips were incubated for an additional hour before the avidity experiment. For the HeLa cells seeded on 18 mm circular glass slides cells were kept overnight in a humidity and CO₂ controlled incubator.

z-Movi® Acoustic Force Avidity Measurements

The effector cells were stained with CellTrace™ Far Red (Thermo Fisher Scientific, cat. #C34564) at 1 μM for 15 minutes in PBS at 37° C. and then resuspended at 10 million/mL in complete medium and used for the avidity experiments. The stained effector cells were introduced in the target cell-seeded flow cell, incubated for 5 minutes, and then a 1 to 1000 pN force ramp was applied throughout 2.5 minutes using a z-Movi® device operated with the Oceon V1.2 software. All cell types were run multiple times (2 runs/sample/chip using two different chips). Samples were run in random order sequentially on the same monolayer. The avidity experiment analysis was performed using Oceon V1.2.

### Centrifuge Experiments

The effector cells were stained with different CellTrace™ dyes (Thermo Fisher Scientific) at 1 μM for 15 minutes in PBS at 37° C. The following stains were used to label the cells with different colors: CAR High: Far Red (cat. #C34564); UNT: Violet (cat. #C34557); CAR unpurified: Yellow (cat. #C34567); CAR Low: CFSE (cat. #C34554). The different cells were mixed in a cell suspension containing equal amounts of the samples for the experiments with the channel slides. Images were acquired with a Nikon Ti2 fluorescence microscope. Data was analysed using NIS Elements software (Nikon).

For experiments using the channel slides the slide was placed into a centrifuge bucket and spun for 2 minutes at 1000 g. This removed the target cells that were not well attached to the glass. The slide was taken out of the centrifuge. 60 μL of effector cell suspension mix was pipetted into the inlet and 60 μl was withdrawn from the outlet, to obtain a homogenous distribution of effector cells along the length of the channel. The slide was incubated upside down for 5 minutes in a humidity and C02 controlled incubator. Three images were taken using the fluorescent microscope, one close to the inlet, one in the center of the channel and one close to the outlet. Each image had Brightfield, FarRed, Violet and Green channels. The slide was put back in the centrifuge and spun at 250 g for 2 min applying a force to the effector cells in a direction away from the target cell layer (as indicated in FIG. 6. The slide was taken out and again three fluorescent images were taken in the same channel positions. The previous step was repeated, increasing the speed to 500 g, 1000 g, 2000 g, 4000 g.

1. A method comprising steps of:
providing a sample holder having a flow channel extending between a channel inlet at an opening side and a channel outlet at the opening side and defining a ceiling associated with the opening side;
loading, in an upright orientation of the sample holder in which the opening side is upward, the flow channel with a liquid containing first cellular objects;
forming a layer of the first cellular objects on the ceiling, by carrying out steps of:
inverting the sample holder into an upside-down orientation, and
incubating the first cellular objects for a defined period in the sample holder while in the upside-down orientation to promote binding of least some of the first cellular objects to the ceiling;
arranging, after the incubating step, the sample holder in the upright orientation, and
subjecting the sample holder to a controllable force for determining an adhesion of the first cellular objects to the ceiling.

2. The method according to claim 1, wherein, during the step of inverting and/or the step of incubating, leaving the channel inlet and/or the channel outlet open during the step of inverting and/or the step of incubating; and wherein the method further comprises selecting the liquid and the channel inlet and/or the channel outlet to retain the liquid in the flow channel by capillary force during the step of inverting and/or the step of incubating.

3. The method according to claim 1, wherein the layer is a first layer and wherein the method further comprises, after the step of arranging, the further steps of:
loading, in the upright orientation, the flow channel with a liquid containing second cellular objects which may interact with the first cellular objects;
forming a second layer of the second cellular objects on the first layer and/or the ceiling, by inverting the sample holder into the upside-down orientation; and
incubating the second cellular objects for a second defined period with the sample holder in the upside-down orientation to promote binding of least some of the second cellular objects to the first layer and/or the ceiling, and
rearranging, after the incubating of the second cellular objects, the sample holder into the upright orientation.

4. The method according to claim 1, further comprising a step of: prior to the step of forming, providing at least part of the ceiling with a functionalisation layer and/or providing at least part of a channel wall with an adhesion prevention layer.

5. The method according to claim 1, further comprising a step of determining an adhesion of the first cellular objects to the ceiling.

6. The method according to claim 65, wherein the step of determining an adhesion includes a further step of applying the controllable force urging at least some of the first cellular objects away from the ceiling.

7. The method according to claim 5, wherein the step of subjecting the sample holder to a controllable force for determining an adhesion comprises applying one or more of a centrifugal force, an acoustic force, a magnetic force, an optical force, and a shear force to the first cellular objects.

8. The method according to claim 1, further comprising a step of removing unbound ones of said first cellular objects from the channel by flushing a liquid through the channel.

9. A sample holder for use in the method according to claim 1, having a flow channel extending between a channel inlet at an opening side and a channel outlet at the opening side and defining a ceiling associated with the opening side, wherein at least part of a channel wall is provided with an adhesion prevention layer.

10. A sample holder according to claim 9, wherein at least part of the ceiling is provided with a functionalisation layer and/or with first cellular objects attached to the ceiling.

11. A sample holder according to claim 9;
wherein the sample holder comprises a liquid reservoir in fluid communication with the channel outlet,
the flow channel defines a channel volume between the channel inlet and the channel outlet for holding a liquid, and
the liquid reservoir defines a reservoir volume for holding an amount of liquid of at least one said channel volume, for holding, when the sample holder is in an upright orientation, a liquid in the flow channel and the reservoir at a liquid level above the channel outlet.

12. The sample holder according to claim 9, containing, in the flow channel, a liquid, wherein a combination of the liquid and the channel inlet and/or the channel outlet are configured to retain the liquid in the flow channel by capillary force when the sample holder is inverted into an upside-down orientation.

13. A sample holder according to claim 11, wherein at least one of
the reservoir surrounds the channel outlet;
the channel outlet is arranged in, and in fluidic communication with, the reservoir; and
the reservoir has an outlet at the opening side larger than the channel inlet and/or channel outlet.

14. The sample holder according to claim 9, comprising a plurality of said flow channels each extending between a respective said channel inlets at an opening side and respective channel outlets at the opening side.

15. The method according to claim 3, wherein the first cellular objects are biological target cells, and the second cellular objects are biological effector cells binding to the target cells.

16. The method according to claim 5, wherein
the at least part of the channel wall is at least part of a floor opposite the ceiling;
the step of determining an adhesion also determines the adhesion of the second cellular objects to the first layer and/or to the ceiling;
the step of applying the controllable force urges at least some of the second cellular objects away from the ceiling;
the step of subjecting the sample holder to the controllable force for determining the adhesion comprises applying one or more of a centrifugal force, an acoustic force, a magnetic force, an optical force, and a shear force to the second cellular objects; and
the step of removes unbound ones of said second cellular objects from the channel by flushing a liquid through the channel.

17. The method according to claim 3, further comprising a step of removing unbound first cellular objects from the channel between the step of arranging the sample holder in the upright orientation and the step of loading the flow channel with a liquid containing ones of said second cellular objects.

18. The sample holder according to claim 11, wherein the at least part of the channel wall includes at least part of a floor opposite the ceiling, the first cellular objects are biological target cells, and the reservoir volume is for holding an amount of liquid of at least three said channel volumes.

19. The sample holder according to claim 13, containing, in the flow channel, a liquid containing biological target cells, wherein a combination of the liquid and the channel inlet and/or the channel outlet are configured to retain the liquid in the flow channel by capillary force when the sample holder is inverted into an upside-down orientation, and the channel outlet is arranged in a floor of the reservoir.

20. The sample hold according to claim 19, comprising a plurality of said flow channels each extending between respective said channel inlets at an opening side and respective channel outlets at the opening side and wherein each flow channel is provided with a respective liquid reservoir in fluid communication with the respective channel outlet.
